(19) United States
(12) Patent Application Publication
(10) Pub. No.: US 2024/0110181 A1
(43) Pub. Date: Apr. 4, 2024

(54) SUBSTANCES AND METHODS FOR TREATING DYSTROPHIC EPIDERMOLYSIS BULLOSA

(71) Applicants: INSTITUT NATIONAL DE LA SANTÉ ET DE LA RECHERCHE MÉDICALE, Paris (FR); UNIVERSITÉ PARIS CITÉ, Paris (FR); FONDATION IMAGINE, Paris (FR); ASSISTANCE PUBLIQUE - HOPITAUX DE PARIS (AP-HP), Paris (FR); SQY THERAPEUTICS, Noisy-Le-Roi (FR); UNIVERSITE DE VERSAILLES ST QUENTIN EN YVELINES, Versailles (FR)

(72) Inventors: Luis GARCIA, Montigny le bretonneux (FR); Aurélie GOYENVALLE, Montigny le bretonneux (FR); Alain HOVNANIAN, Paris (FR); Matthias TITEUX, Paris (FR)

(21) Appl. No.: 18/266,034

(22) PCT Filed: Dec. 9, 2021
(86) PCT No.: PCT/EP2021/084959

§ 371 Date: Jun. 8, 2023

(30) Foreign Application Priority Data
Dec. 10, 2020 (EP) 20306530.5

(51) Int. Cl. C12N 15/113

(52) U.S. Cl. CPC C12N 15/113; C12N 2310/321; C12N 2310/3231; C12N 2310/3515; C12N 2320/33

(57) ABSTRACT

Epidermolysis bullosa (EB) is caused by mutations in the COL7A1 gene encoding type VII collagen. EB comprises a phenotypically diverse group of inherited blistering diseases that affect the skin, and possibly mucous membranes and other organs A number of therapeutic strategies have been explored for managing the treatment of EB, such as (i) surgery, (ii) chemotherapy, (iii) use of collagenase activity inhibitors, (iv) use of antibiotics, (v) use of anti-inflammatory compound, (vi) cell therapies, (viii) protein therapy as well as (ix) gene therapy. Gene therapy of EB aimed to provide therapeutic benefit through manipulation of DNA or RNA. Anti-sense therapy targeting specific exons have been described, such as targeting exons 73 and 80. However, this kind of therapeutic strategy had to be improved as regards its pharmaceutical compliance. The present disclosure provides an improved antisense therapeutic strategy targeting exon 73 of COL7A1, which includes the conception of novel tricyclo-DNA antisens nucleic acids, which, in some embodiments, may be conjugated to lipid moieties.

### FIELD OF THE DISCLOSURE

The present disclosure concerns the medical field relating to the treatment of dystrophic epidermolysis bullosa.

### BACKGROUND OF THE DISCLOSURE

Epidermolysis bullosa (EB) comprises a phenotypically diverse group of inherited blistering diseases that affect the skin and, in some subtypes, mucous membranes and other organs. Clinically, individuals with EB have fragile skin and are susceptible to blistering following minimal trauma. The sub-classification of EB extends to over 30 clinical subtypes with pathogenic mutations in at least 21 distinct genes.

DEB is caused by mutations in the COL7A1 gene, encoding type VII collagen (C7) the constituent of anchoring fibrils, which form essential structures for dermal-epidermal adhesion.DEB can be inherited either in an autosomal dominant or autosomal recessive pattern with extensive variability in the clinical phenotype. Skin detachments and blisters, either spontaneous or secondary to minimal trauma, develop into chronic and extensive wounds with systemic complications especially in the generalized recessive forms. All mucous membranes (oral, esophageal, ocular, genital, anal) can also be affected.

A number of therapeutic strategies have been explored for managing the treatment of both DDEB and RDEB, which encompass (i) surgery, (ii) chemotherapy in the case of occurrence of squamous cell carcinoma, systemic treatment with (iii) collagenase activity inhibitor (e.g. phenytoin), (iv) antibiotics (e.g. minocycline, trimethoprim), (v) anti-inflammatory compounds (e.g. ciclosporin), (vi) cell therapies, such as involving allogenic fibroblasts, mesenchymal stromal cells, bone marrow transplantation, grafting revertant mosaicism skin/keratinocytes, (viii) protein therapy, based on intradermal injections of recombinant C7, as well as (ix) gene therapy.

Gene therapy strategies in RDEB aim to provide therapeutic benefit through manipulation of DNA or RNA. Typically, viral mediated ex vivo gene transfer approaches have been used whereby the patient's skin cells are cultured, transduced with a viral vector encoding the transgene expressing the wild-type protein and then these genetically modified cells can either be transplanted back via grafting of epithelial sheets or skin equivalents (epidermis/dermis), or by intradermal injections (e.g. of genetically supplemented fibroblasts). Gene silencing technologies such as RNA interference (RNAi) are useful in dominant forms of DEB, if designed to knockdown the mutant allele without silencing the wild-type allele, with pre-clinical data to support therapeutic use of such an approach (Pendaries et al., 2012, J Invest Dermatol, Vol. 132: 1741-1743; Morgan et al., 2013, Vol. 133: 2793-2796). Another methodology, relevant both for RDEB and DDEB, has been trying to modulate splicing of pre-messenger RNA to induce skipping of the mutated exon. Illustratively, a COL7A1 minigene lacking exons 70-104 encodes de protein that is able to trimerize in vitro (Chen et al., 2000, J Biol Chem, Vol. 275: 24429-24435). Transfer of this minigene to RDEB cells restored a wild-type phenotype in cell migration assays.

Exons 73 and 80 were identified in the art as of particular interest, because they carry many recurrent recessive and dominant mutations. Exon 73 carries the largest number of variations, and about 7.5% of RDEB patients harbor at least one mutation in exon 73 (Van den Akker et al., 2011, Hum Mutat, Vol. 32: 1100-1107). Exon 80, although displaying fewer variations, carries a recurrent mutation (c.6527insC) with a founder effect accounting for 46% of RDEB alleles in Spain (Escamez et al., 2010, Br J Dermatol, Vol. 163: 155-161) and 42% in the Chilean population (Rodriguez et al., 2012, J Dermatol Sci, Vol. 65: 149-152). Using 2′-O-methyl antisense oligoribonucleotides (AONs) in an RDEB skin equivalent xenograft model, one or two subcutaneous injections of AONs at doses ranging from 400 lag up to 1 mg was able to induce skipping of exons containing loss-of-function mutations in exons 73 and 80 and thereby restore C7 expression and anchoring fibril formation (Turczynski et al., 2016, Vol. 136: 2387-2395).

There remains a need in the art for providing therapeutic tools, alternative or improved as regards the known therapeutic strategies, for treating dystrophic epidermolysis bullosa.

### SUMMARY OF THE DISCLOSURE

The present disclosure relates to a nucleic acid consisting of N consecutive nucleotides of the nucleic acid sequence of SEQ ID NO. 1 below:

(SEQ ID NO. 1)
5′-GCCCGCGTTCTCCAG-3′,

wherein,
N is an integer ranging from 13 to 15
N-x nucleotides consist of tricyclonucleotides, with x being an integer equal to 1 or 2,
x nucleotides consist of 2′-O-methyl-ribonucleotides, and
each nucleotide is linked to an adjacent nucleotide through a phosphodiester internucleoside linkage

In some embodiments of the said nucleic acid, the tricyclonucleotide consists of a compound of formula (I) as described in the present disclosure.

In some embodiments, the said nucleic acid comprises one 2′-O-methyl-ribonucleotide.

In some embodiments of the said nucleic acid, (i) N is 15, (ii) x means 1, (iii) all internucleoside linkages consist of phosphodiester linkages, and (iv) the said nucleic acid comprises one nucleotide consisting of 2′-O-methyl-ribonucleotide.

In some embodiments, the said nucleic acid consists of the oligonucleotide of SEQ ID NO. 1 as described in the present disclosure, wherein: (i) all internucleoside linkages consist of phosphodiester linkages, and (ii) all the nucleotides consist of tricyclonucleotides, excepted the cytosyl nucleotide located at position 4, in the sense from the 5′-end to the 3′-end, which consists of a 2′-O-Methyl ribocytosyl.

In some embodiments, the said nucleic acid comprising one or more lipid moieties covalently linked thereto, such as a lipid moiety selected from the group consisting of a saturated fatty acid moiety and an unsaturated fatty acid moiety.

In some embodiments, the 5′ end nucleotide of the said nucleic acid is covalently linked to a palmitoyl group.

The present disclosure also pertains to a vector comprising a nucleic acid as described in the present disclosure.

This disclosure also concerns a pharmaceutical composition comprising a nucleic acid as described herein, or a vector as described herein, and a pharmaceutically acceptable vehicle.

### DESCRIPTION OF THE FIGURES

FIG. 1 illustrates skipping efficiency of various antisense nucleic acids aimed at causing the skipping of exon 73 of human COL7A1 gene. Ordinate: exon skipping efficiency values. Abscissa: names of the tested antisense nucleic acids.

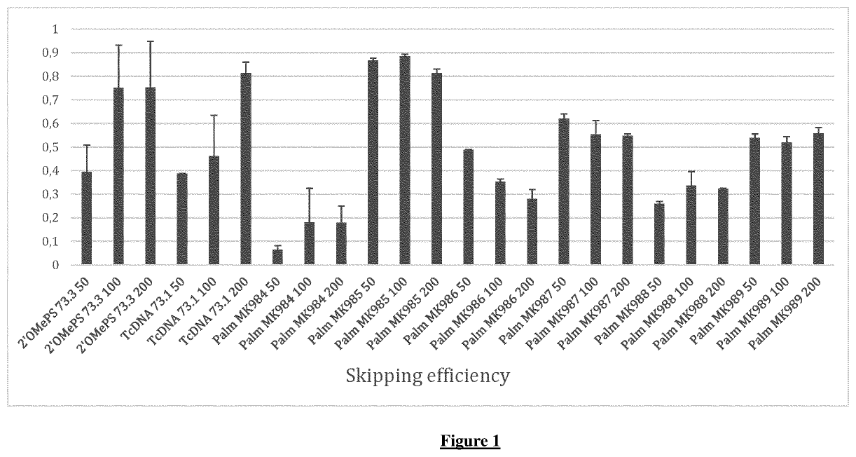

### DETAILED DESCRIPTION

The present description discloses methods for restoring the function of mutated type VII collagen protein using exon skipping technology. The method involves blocking or preventing the incorporation into mature mRNA of mutated exon 73 of COL7A1 carrying dominant or recessive mutations responsible for collagen type VII dysfunction. This is accomplished by exposing the pre-mRNA that includes exons encoding the protein to antisense oligonucleotides (AONs) which are complementary to sequence motifs that are required for correct splicing of the targeted exon 73 in the COL7A1 pre-mRNA. The AONs bind to complementary required sequences in the pre-mRNA and prevent normal splicing. Instead, the targeted exon is excised and is not included in the mature mRNA that is translated into protein, and the amino acid sequence encoded by the targeted exon is missing from the translated protein

### Definitions

The terms used in this specification generally have their ordinary meanings in the art. Certain terms are discussed below, or elsewhere in the present disclosure, to provide additional guidance in describing the products and methods of the presently disclosed subject matter.

The definitions below apply in the context of the present disclosure.

As used in this specification and the appended claims, the singular forms “a”, “an” and “the” include plural referents unless the content clearly dictates otherwise.

It is understood that aspects and embodiments of the present disclosure described herein include “having,” “comprising,” “consisting of,” and “consisting essentially of” ‘ aspects and embodiments. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” ’ implies the inclusion of the stated element(s), to the exclusion of any additional elements. The term “consisting essentially of” implies the inclusion of the stated elements, and possibly other element(s) where the other element(s) do not materially affect the basic and novel characteristic(s) of the invention.

The term “antisense oligonucleotide” refers to a single strand of DNA or RNA that is complementary to a selected nucleic acid sequence.

“Exon skipping” generally refers to the removal of an entire exon or part thereof from a selected pre-processed RNA and, as a result, is excluded from mature RNA (such as translation into protein mature mRNA). Therefore, there is no protein portion in the expressed protein form that is additionally encoded by the skipped exon, and usually forms a modified but still functional protein form.

«COL7A1 » designates the gene encoding the human type VII collagen. For COL7A1, nucleic acid sequence, it may be referred to OMIM*120120 at the website address www.omim.org.

The term “nucleobase” means any nitrogen-containing heterocyclic moiety capable of forming Watson-Crick-type hydrogen bonds and stacking interactions in pairing with a complementary nucleobase when that nucleobase is incorporated into a polymeric structure.

The terms “internucleoside linkage,” “internucleoside linking group,” “internucleotide linkage”, or “internucleotide linking group” are used herein interchangeably and refer to any linker or linkage between two nucleoside (i.e., a heterocyclic base moiety and a sugar moiety) units, as is known in the art. A “internucleoside linking group” may be involved in the linkage between two nucleosides, between two nucleoside analogs or between a nucleoside and a nucleoside analog. Internucleoside linkages constitute the backbone of a nucleic acid molecule. An internucleoside linking group refers to a chemical group linking two adjacent nucleoside residues comprised in a nucleic acid molecule, which encompasses (i) a chemical group linking two adjacent nucleoside residues, (ii) a chemical group linking a nucleoside residue with an adjacent nucleoside analog residue and (iii) a chemical group linking a first nucleoside analog residue with a second nucleoside analog residue, which nucleoside analog residues may be identical or may be distinct. A 3′-5′ internucleoside linkage, as used herein, refers to an internucleoside linkage that links two adjacent nucleoside units, wherein the linkage is between the 3′-carbon of the sugar moiety of the first nucleoside and the 5′-carbon of the sugar moiety of the second nucleoside.

Phosphodiester internucleoside linkages are internucleoside linkages naturally found in RNA as well as DNA and involves the phosphate group and 3′- and 5′-hydroxyl groups of the pentose (PO) sugar moiety thereof.

Phosphorothioate (PS) backbones are the most largely used chemical modifications to protect antisense oligonucleotides (AONs) from nuclease activity and increase their stability to target RNA (Eckstein, 2014, Nucleic Acid Therapeutics, Vol. 24 (6): 374-387). Typical PS bonds differ from phosphodiester (PO) bonds by the non-bridging phosphate 0-atoms being replaced with a S-atom which confers higher stability and increased cellular uptake (Benimetskaya et al., 1995, Nucleic Acids Research, Vol. 23: 4239-4245). PS modifications have demonstrated an elevated efficacy due to an increased bioavailability compared to their PO counterparts (Matsukura et al., 1987, Proc Natl Acad Sci USA, Vol. 84: 7706-7710) and most of the drugs currently under clinical programs include PS bonds (Crooke et al, 2020, J Am Chem Soc, Vol. 142 (35): 14754-14771).

The term “lipid moiety” as used herein refers to moieties that are derived from, typically and preferably, hydrocarbons, oils, fats (such as fatty acids, glycerides), sterols, steroids, and derivative forms of these compounds. Suitable lipid moieties include moieties derived from fatty acids and their derivatives, hydrocarbons and their derivatives, and sterols, such as cholesterol. As used herein, the term lipid moiety also includes amphipathic compound moieties, which contain both lipid and hydrophilic moieties.

The term “fatty acid”, as used herein, refers to a hydrocarbon chain that terminates with a carboxylic acid group, wherein said hydrocarbon chain is typically and preferably either an alkyl or alkenyl of typically 6 to 32 carbons long, and that are, thus, saturated or unsaturated.

The term “fatty acid moiety”, as used herein, refers to a moiety derived from a fatty acid, as defined herein, wherein one carboxylic group (—COOH) of said fatty acid becomes and is a —C(O)— group of said fatty acid moiety, which —C(O)— group is linked to said oligonucleotide either directly or indirectly.

The term “alkyl”, as used herein, refers to a straight or branched hydrocarbon chain radical consisting solely of carbon and hydrogen atoms, containing no unsaturation, having a specified number of carbon atoms (e.g., (C 6-32)alkyl or C 6-32 alkyl), and which may be or typically is attached to the rest of the molecule by a single bond.

In its broadest meaning, the term “treating” or “treatment” refers to reversing, alleviating, inhibiting the progress of, or preventing the disorder or condition to which such term applies, or one or more symptoms of such disorder or condition. In the context of the present disclosure, the terms “treat”, “treatment”, and the like refer especially to Dystrophic Epidermolysis Bullosa.

As used herein, the terms “therapeutically effective amount” and “prophylactically effective amount” refer to an amount that provides a therapeutic benefit in the treatment, prevention, or management of pathological processes mediated by expression of a mutated gene, especially by expression of COL7A1 mutated in exon 73. The specific amount that is therapeutically effective can be readily determined by an ordinary medical practitioner, and may vary depending on factors such as the type and stage of pathological processes mediated by the target gene expression, the patient's medical history and age, and the administration of other therapeutic agents that inhibit biological processes mediated by the mutated gene.

As used herein, the term “individual” or “subject” is a mammal, most preferably a human. Mammals include, but are not limited to, domesticated animals (e.g., cows, sheep, cats, dogs, and horses), primates (e.g., humans and non-human primates such as monkeys), rabbits, and rodents (e.g., mice and rats). Most preferably, the individual or subject is a human.

### DETAILED DISCLOSURE

The inventors have conceived a family of nucleic acids of a short length targeting exon 73 of the human COL7A1 gene, that efficiently induces skipping of exon 73, which nucleic acids are useful for treating human subjects bearing a COL7A1 gene that is mutated in exon 73 and wherein the presence of one or more mutations in the said exon causes diseases, and especially causes Dystrophic Epidermolysis Bullosa.

Nucleic acids according to the present disclosure may also be termed “antisense nucleic acids” herein.

As it will be illustrated throughout the present disclosure, the nucleic acids described herein have significantly improved properties as compared with the antisense polynucleotides also targeting exon 73 of the human COL7A1 gene that have been previously described in the art.

After a thorough research, the inventors have succeeded in conceiving human COL7A1 exon 73-targeting nucleic acids that efficiently induce exon 73 skipping and which have a short nucleotide length.

According to the inventors knowledge, previously known antisense nucleic acids targeting COL7A1 exon 73 were of about from 25 to 30 nucleotides in length,

In contrast, the present disclosure relates to COL7A1 exon 73-targeting antisense nucleic acids which are of at most 15 nucleotides in length.

Thus, the inventors have succeeded in conceiving nucleic acids that are of a very short nucleotide length while possessing a high specificity for the targeted mRNA sequence and also a high stability of hybridization with the said targeted sequence, which explains why, unexpectedly, such nucleic acids of a short nucleotide length cause an efficient skipping of human COL7A1 exon 73, as shown in the examples herein.

The present disclosure relates to a nucleic acid consisting of N consecutive nucleotides of the nucleic acid sequence of SEQ ID NO. 1 below:

(SEQ ID NO. 1)
5′-GCCCGCGTTCTCCAG-3′,

wherein,
N is an integer ranging from 13 to 15
N-x nucleotides consist of tricyclonucleotides, with x being an integer equal to 1 or 2,
x nucleotides consist of 2′-O-methyl-deoxyribonucleotides, and
each nucleotide is linked to an adjacent nucleotide through a phosphodiester internucleoside linkage.

The fact that the nucleic acids described herein have a short nucleotide length allows their good distribution in the tissues upon their administration to a subject in need thereof, as compared with longer nucleic acids. The good distribution properties of the nucleic acids described herein will indeed significantly contribute to their high therapeutic efficiency, as compared with longer nucleic acids.

Further, by definition, short nucleic acids exhibit a significantly lower toxicity than longer nucleic acids. Consequently, the nucleic acids according to the present disclosure, when they are administered to a subject in need thereof, are less toxic than the longer antisense nucleic acids aimed at inducing the skipping of COL7A1 exon 73.

Incidentally, producing therapeutically active short nucleic acids is also less expensive than synthesizing longer nucleic acids.

Unexpectedly, the nucleic acids described herein are not prone to enzymatic cleavage, although the nucleotides comprised therein all consist of phosphodiester bonds, which phosphodiester bonds are known in the art to be enzyme-sensitive.

Most of the nucleotides of a nucleic acid as described herein consist of tricyclonucleotides, which feature contributes to high hybridization properties to the targeted mRNA sequence, which encompasses both binding with high affinity and binding with high selectivity to complementary RNA. Further, tricyclonucleotides do not elicit RNaseH activity and also exhibit remarkable stability in serum.

Further, as described above, a nucleic acid disclosed herein comprises one or two 2′-O-methyl-deoxyribonucleotides. The presence of one or two 2′-O-methyl-deoxyribonucleotides allows reducing the tendency of the nucleic acid to form homodimers and thus also contributes to its exon skipping efficacy. Without wishing to be bound by any particular theory, the inventors believe that the presence of one or two 2′-O-methyl-deoxyribonucleotides also (i) improves the biodistribution of the nucleic acid in vivo, and (iii) reduces undesirable immune stimulation as well as other non-specific effects.

Although phosphorothioate internucleoside linking groups are known in the art for their advantageous properties of facilitating nucleic acid biodistribution, the present inventors have excluded their presence in a nucleic acid as described herein.

Noticeably, the nucleic acids described herein stably bind to the target RNA and are protected from nuclease activity although these nucleic acids do not comprise any phosphorothioate internucleoside linkage. Without wishing to be bound by any particular theory, the inventors believe that the absence of a phosphorothioate internucleoside linkage contributes to the reduced toxicity of the nucleic acids described herein.

Indeed, tricylonucleotides are well known in the art and have been already used for manufacturing antisense oligonucleotides for therapeutic splice-switching correction in many genetic diseases, which include Duchenne muscular dystrophy (See for illustration Goyenvalle et al., 2016, J Neuromuscul Dis. 2016 May 27;3(2):157-167). Tricyclonucleotides and methods for manufacturing tricyclonucleotides are notably described by Steffens and Leumann, Journal of the American Chemical Society 1999 121 (14), 3249-3255 According to preferred embodiments of the present disclosure, a tricyclonucleotide consists of a compound of the formula (I) below:

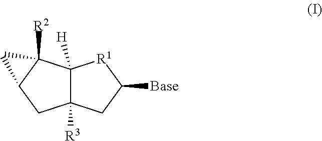

wherein
R1 means 0, S, CH 2 or NR where R is hydrogen or a (C1-C3) non-substituted alkyl,
each of R2 and R3, one independently from the other, means an internucleoside linkage, and
“Base” means a nucleobase.

In most preferred embodiments of a tricyclonucleotide of formula (I), R1 means 0. According to these most preferred embodiments, R2 and R3, one independently from the other, mean internucleoside linkages.

In a tricyclonucleotide of formula (I) according to the present disclosure, the nucleobase is selected in the group consisting of Adenine (A), Thymine (T), Guanine (G) and Cytosine, i.e. the nucleobases that are represented in the nucleotides comprised in the nucleic acid of SEQ ID NO. 1.

A further preferred feature of a nucleic acid according to the present disclosure is the presence of one or two 2′-O-methyl ribonucleotides therein. Unexpectedly, the presence of 2′-O-methyl ribonucleotides in a nucleic acid as described herein does not significantly alter its ability to hybridize to its target sequence in exon 73 of COL7A1 mRNA, and improves its biodistribution and safety profile.

In most preferred embodiments, a nucleic acid according to the present disclosure comprises only one 2′-O-methyl ribonucleotide.

As it is shown in the examples herein, all the nucleic acids according to the present disclosure and comprising a single 2′-O-methyl ribonucleotide efficiently induce the COL7A1 exon 73 skipping which is sought, since the whole exemplified nucleic acids induce exon skipping with 50% efficacy or more.

As it is also shown in the examples, the location of the said single 2′-O-methyl ribonucleotide may contribute both (i) to the level of exon skipping efficacy and (ii) to the ability of the nucleic acid to form homodimers.

Preferred nucleic acids according to the present disclosure are those for which (i) the integer N is 15, (ii) the integer x means 1, (iii) all internucleoside linkages consist of phosphodiester linkages and (iv) the said nucleic acid comprises one nucleotide consisting of 2′-O-methyl-ribonucleotide (i.e. the sole nucleotide that does not consist of a tricyclonucleotide).

As illustrated herein, optimal nucleic acids according to the present disclosure are those for which the single 2′-O-methyl deoxyribonucleotide is located at a nucleotide position selected in the group consisting of nucleotide positions 4 (C), 6 (C) and 7 (G) of SEQ ID NO. 1, according to a nucleotide numbering sense from 5′-end to 3′-end.

The nucleic acid of SEQ ID NO. 1 comprising a single 2′-O-methyl deoxyribonucleotide at position 4 (C) is also referenced as the nucleic acid of SEQ ID NO. 2 herein.

The nucleic acid of SEQ ID NO. 1 comprising a single 2′-O-methyl deoxyribonucleotide at position 6 (C) is also referenced as the nucleic acid of SEQ ID NO. 4 herein.

The nucleic acid of SEQ ID NO. 1 comprising a single 2′-O-methyl deoxyribonucleotide at position 7 (G) is also referenced as the nucleic acid of SEQ ID NO. 5 herein.

Most preferred nucleic acids according to the present disclosure consist of the oligonucleotide of SEQ ID NO. 1, wherein:
all internucleoside linkages consist of phosphodiester linkages,
all the nucleotides consist of tricyclonucleotides, excepted one nucleotide which consists of a 2′-O-methyl-ribonucleotide selected in the group consisting of (i) the cytosyl nucleotide at position 4, (ii) the cytosyl nucleotide at position 6 and (iii) the guanyl nucleotide at position 7, in the sense from 5′-end to 3′-end.

The most preferred nucleic acid according to the present disclosure consists of the oligonucleotide of SEQ ID NO. 1, wherein:
all internucleoside linkages consist of phosphodiester linkages,
all the nucleotides consist of tricyclonucleotides, excepted the cytosyl nucleotide located at position 4, in the sense form the 5′-end to the 3′-end, which consists of a 2′-O-methyl-deoxyribocytosyl.

As already previously mentioned herein, most preferred tricyclonucleotides are those of formula (I) wherein R1 means 0, and R1, R2 and “Base” are as defined for formula (I).

Thus, at least in the above-described nucleic acids for which the location of the single 2′-O-methyl nucleotide is specified, the tricyclonucleotides comprised therein are tricyclonucleotides of formula (I) wherein (i) R1 is 0 and (ii) each of R2 and R3 means a phosphodiester linkage and (iii) “Base” means a nucleobase.

Nucleic Acid Synthesis

Numerous methods for synthesizing nucleic acids are well known in the art for decades, to which the skilled artisan may easily refer.

For use according to the present disclosure, the nucleic acids as described herein can be synthesized de novo using any of a number of procedures well known in the art. For example, the b-cyanoethyl phosphoramidite method; nucleoside H-phosphonate method. These chemistries can be performed by a variety of automated nucleic acid synthesizers available in the market. These nucleic acids may be referred to as synthetic analogue nucleic acids.

Conjugated Nucleic Acids

In preferred embodiments of a nucleic acid according to the present disclosure, the said nucleic acid is conjugated to, i.e. covalently linked to, one or more lipid moieties. Conjugation of a nucleic acid to lipid moieties may be performed with a variety of known methods (Benizri et al., 2019, Bioconjugate Chemistry, American Chemical Society, Vol. 30: 366-383-(https://hal.archives-ouvertes.fr/hal-02490446)).

As it is known in the art, conjugation of an antisense nucleic acid to a lipid moiety allows an increased tissue targeting of the said antisense nucleic acid increases its efficacy. In the present context, conjugating an antisense nucleic acid as described herein to one or more lipid moieties increases its tissue targeting ability and thus enhances its efficiency in COL7A1 exon 73 skipping.

Palmitic acid is known to bind to albumin and conjugation of palmitic acid to PS ASO is expected to improve albumin binding and therefore increase bioavailability of palmitate-conjugated ASOs (Prakash et al., 2019, Nucleic Acids Research, Vol. 47 (12): 6029-6044).

In some preferred embodiments, a single lipid moiety is covalently linked, either directly or via a linker group, to a nucleic acid as described herein.

Most preferably, the one or more lipid moieties are covalently linked to the 5′-end nucleotide and/or to the 3′-end nucleotide by a phosphoramidite bond.

In some embodiments, a nucleic acid according to the present disclosure and conjugated to a lipid moiety has a structure of formula (I) below (illustrated by a lipid moiety linked to the 5′-end nucleoside):

Lipid-[Linker]z-NuclAc (I), wherein
“Lipid” means a lipid moiety,
“Linker” means a chemical linker allowing, when present, to covalently link the lipid moiety to the nucleic acid according to the present disclosure,
z is an integer meaning 0 or 1 and
NuclAc means a nucleic acid according to the present disclosure.

In preferred embodiments, the said one or more lipid moieties consist of one or more fatty acids.

be designated according to their “C:D” value, wherein “C” is the total number of carbons comprised therein and “D” is the number of unsaturated bonds comprised therein.

According to the present disclosure, fatty acids may be selected in a group saturated fatty acids comprising caproic acid (C6:0), caprylic acid (C8:0), capric acid (C10:0) and lauric acid (C12:0), palmitic acid (C16:0), stearic acid (C18:0), arachidic acid (C20:0), behenic acid (C22:0), lignoceric acid and cerotic acid (C26:0).

In some embodiments, fatty acids may be selected in a group of unsaturated fatty acids comprising myristoleic acid (C14:1), palmitoleic acid (C16:1), oleic acid (C18:1), linoleic acid (C18:2), arachidonic acid (C20:4), eicosapentaenoic acid (C20:5) and docosahexaenoic acid (C22:6).

In most preferred embodiments, the said one or more lipid moieties consist of palmitic acid (i.e. which becomes a palmitoyl group when covalently linked, either directly of via a linker group” to the selected nucleic acid).

In preferred embodiments, a single fatty acid moiety is covalently linked, either directly or via a linker group, to a nucleic acid according to the present disclosure, the said single fatty acid moiety being most preferably covalently linked to the nucleotide located at the 5′-end of the said nucleic acid.

Most preferred embodiments of a conjugated nucleic acid as described herein are those wherein a single palmitoyl group is covalently linked, either directly or via a linker group, to the nucleotide located at the 5′-end of the said nucleic acid.

In some embodiments, such a conjugated nucleic acid is illustrated by the formula (II) below:

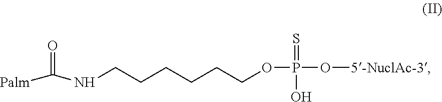

wherein,
“Palm-(C=0)-” means a palmitoyl moiety, and
“NuclAc” means a nucleic acid according to the present disclosure.

A conjugated nucleic acid of formula (II) above may also be termed “Palm-C6-amino-* nucleic acid” herein. As it is readily understood, a conjugated nucleic acid conjugate of formula (II) is an embodiment of a conjugated amino acid of formula (I) wherein:
“Lipid” means a palmitoyl moiety, and
z means 1 and “Linker” means a C6 alkyl group.

Therapeutic Uses

The nucleic acids described herein are used to cause exon 73 of COL7A1 mRNA skipping, resulting in an amelioration of Dystrophic Epidermolysis Bullosa symptoms (i.e. restoration of protein function or stability) as compared with a non-treated patient case. Such symptoms may be observed on a micro level (i.e. restoration of protein expression and/or localisation evaluated by immunohistochemistry, immunofluorescence, western-blot analyses); amelioration of the skin lesion by histological examination; restoration/amelioration of protein functionality evaluated by the ability to form anchoring fibril between the external epithelia and the underlying stroma.

Antisense nucleic acids according to the present disclosure may be delivered in vivo alone or in association with a vector. In its broadest sense, a “vector” is any vehicle capable of facilitating the transfer of the antisense nucleic acid to the cells and preferably cells expressing collagen VII.

Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense nucleic acids. Viral vectors are a preferred type of vector and include, but are not limited to nucleic acid sequences from the following viruses: lentivirus such as HIV-1, retrovirus, such as moloney murine leukemia virus, adenovirus, adeno-associated virus; SV40-type viruses; Herpes viruses such as HSV-1 and vaccinia virus. One can readily employ other vectors not named but known to the art. Among the vectors that have been validated for clinical applications and that can be used to deliver the antisense nucleic acids, lentivirus, retrovirus and AAV show a greater potential for exon skipping strategy.

Retrovirus-based and lentivirus-based vectors that are replication-deficient (i.e., capable of directing synthesis of the desired proteins, but incapable of manufacturing an infectious particle) have been approved for human gene therapy trials. They have the property to integrate into the target cell genome, thus allowing for a persistent transgene expression in the target cells and their progeny.

The human parvovirus Adeno-Associated Virus (AAV) is a dependovirus that is naturally defective for replication which is able to integrate into the genome of the infected cell to establish a latent infection. The last property appears to be unique among mammalian viruses because the integration occurs at a specific site in the human genome, called AAVS1, located on the chromosome 19 (19q13.3—qter). AAV-based recombinant vectors lack the Rep protein, AAV vectors and integrate with low efficacy and low specificity into the host genome, and are mainly present as stable circular episomes that can persists for months and maybe years in the target cells. Therefore AAV has aroused considerable interest as a potential vector for human gene therapy. Among the favourable properties of the virus are its lack of association with any human disease and the wide range of cell lines derived from different tissues that can be infected. Actually 12 different AAV serotypes (AAV1 to 12) are known, each with different tissue tropisms (Wu et al., 2006). Nonetheless, AAV are very valuable vectors which are now extensively used to transfer small antisens sequences to selectively knock-down alleles or modulate the splicing of target genes (Goyenvalle et al., 2004; Xia et al., 2004).

Other vectors include plasmid vectors. Plasmid vectors have been extensively described in the art and are well known to those skilled in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. Plasmids may be delivered by a variety of parenteral, mucosal and topical routes. For example, the DNA plasmid can be injected by intradermal, subcutaneous, or other routes. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the vectorized antisense sequences are fused with a small nuclear RNA (snRNA) such as U7 or Ul in order to ensure their stability and spliceosome targeting (Goyenvalle et al., 2004; Montgomery and Dietz, 1997).

A further object of the present disclosure relates to a method for the treatment of a patient suffering from DEB comprising the step of administering to the said patient a nucleic acid as described herein, most preferably a conjugated nucleic acid as described herein. According to the present disclosure, exon 73 of COL7A1 mRNA is removed upon administration of a nucleic acid as described herein, in order to restore the functionality of a mutated collagen VII.

In a more particular embodiment, said antisense oligonucleotides are those depicted in Table 2 and may be associated with a vector as above described.

The invention further relates to a nucleic acid as described herein, or a vector comprising the said nucleic acid, for use in the treatment of Dystrophic Epidermolysis Bullosa.

The present invention also provides a pharmaceutical composition comprising a nucleic acid as described herein, or a vector comprising the said nucleic acid, for use in the treatment of a Dystrophic Epidermolysis Bullosa.

In addition to a nucleic acid according to the present disclosure, or to a vector comprising the said nucleic acid, pharmaceutical compositions may also include a pharmaceutically or physiologically acceptable carrier such as saline, sodium phosphate, etc. The compositions will generally be in the form of a liquid, although this need not always be the case. Suitable carriers, excipients and diluents include lactose, dextrose, sucrose, sorbitol, mannitol, starches, gum acacia, calcium phosphates, alginate, tragacanth, gelatin, calcium silicate, microcrystalline cellulose, polyvinylpyrrolidone, celluose, water syrup, methyl cellulose, methyl and propylhydroxybenzoates, mineral oil, etc. The formulations can also include lubricating agents, wetting agents, emulsifying agents, preservatives, buffering agents, etc. In particular, the present disclosure involves the administration of nucleic acids, or of vectors comprising them, and is thus somewhat akin to gene therapy. Those skilled in the art will recognize that nucleic acids are often delivered in conjunction with lipids (e.g. cationic lipids or neutral lipids, or mixtures of these), frequently in the form of liposomes or other suitable micro- or nano-structured material (e.g. micelles, lipocomplexes, dendrimers, emulsions, cubic phases, etc.).

The pharmaceutical compositions according to the present disclosure are generally administered by injection, e.g. intravenously, subcutaneously or intramuscularly. In some embodiments, topical administration of the composition may be performed.

Injectable preparations, for example, sterile injectable aqueous or oleaginous suspensions may be formulated according to the known art using suitable dispensing or wetting agents and suspending agents. The sterile injectable preparation can also be a sterile injectable solution or suspension in a nontoxic parenterally acceptable diluent or solvent, for example, as a solution in 1,3-butanediol.

In a particular embodiment, it may be desirable to administer a nucleic acid as described herein, or a vector comprising the said nucleic acid, in admixture with a topical pharmaceutically acceptable carrier. The topical pharmaceutically acceptable carrier is any substantially nontoxic carrier conventionally usable for topical administration of pharmaceuticals in which a nucleic acid as described herein, or a vector comprising it, will remain stable and bioavailable when applied directly to skin surfaces. For example, carriers such as those known in the art effective for penetrating the keratin layer of the skin into the stratum corneum may be useful in delivering the nucleic acids of the present disclosure or a vector comprising them, to the area of interest. Such carriers include liposomes. A nucleic acid according to the present disclosure, or a vector comprising the said nucleic acid, can be dispersed or emulsified in a medium in a conventional manner to form a liquid preparation or mixed with a semi-solid (gel) or solid carrier to form a paste, powder, ointment, cream, lotion or the like.

Suitable topical pharmaceutically acceptable carriers include water, buffered saline, petroleum jelly (vaseline), petrolatum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, synthetic polymers, alcohols, polyols, and the like. The carrier can be a water miscible carrier composition. Such water miscible, topical pharmaceutically acceptable carrier composition can include those made with one or more appropriate ingredients outset of therapy.

It may be desirable to have a delivery system that controls the release of antisense oligonucleotides of the invention to the skin and adheres to or maintains itself on the skin for an extended period of time to increase the contact time of a nucleic acid of the present disclosure, or a vector comprising the said nucleic acid, on the skin. Sustained or delayed release of antisense oligonucleotides provides a more efficient administration resulting in less frequent and/or decreased dosage of antisense oligonucleotides and better patient compliance. Examples of suitable carriers for sustained or delayed release in a moist environment include gelatin, gum arabic, xanthane polymers. Pharmaceutical carriers capable of releasing the antisense oligonucleotides of the invention when exposed to any oily, fatty, waxy, or moist environment on the area being treated, include thermoplastic or flexible thermoset resin or elastomer including thermoplastic resins such as polyvinyl halides, polyvinyl esters, polyvinylidene halides and halogenated polyolefins, elastomers such as brasiliensis, polydienes, and halogenated natural and synthetic rubbers, and flexible thermoset resins such as polyurethanes, epoxy resins and the like. Controlled delivery systems are described, for example, in U.S. Pat. No. 5,427,778 which provides gel formulations and viscous solutions for delivery of the antisense oligonucleotides of the invention (or a vector comprising thereof) to a skin site. Gels have the advantages of having a high water content to keep the skin moist, the ability to absorb skin exudate, easy application and easy removal by washing. Preferably, the sustained or delayed release carrier is a gel, liposome, microsponge or microsphere. The antisense oligonucleotides of the invention (or a vector comprising thereof) can also be administered in combination with other pharmaceutically effective agents including, but not limited to, antibiotics, other skin healing agents, and antioxidants.

One skilled in the art will recognize that the amount of a nucleic acid according to the present disclosure, or a vector comprising the said nucleic acid, to be administered will be an amount that is sufficient to induce amelioration of unwanted disease symptoms. Such an amount may vary inter alia depending on such factors as the gender, age, weight, overall physical condition, of the patient, etc. and may be determined on a case by case basis. The amount may also vary according to the type of condition being treated, and the other components of a treatment protocol (e.g. administration of other medicaments such as steroids, etc.). Generally, a suitable dose is in the range of from about 1 mg/kg to about 100 mg/kg. If a viral-based delivery of AONs is chosen, suitable doses will depend on different factors such as the viral strain that is employed, the route of delivery (intramuscular, intravenous, intra-arterial or other), but may typically range from 10¹⁰ to 10¹² viral particles/kg. Those skilled in the art will recognize that such parameters are normally worked out during clinical trials. Further, those skilled in the art will recognize that, while disease symptoms may be completely alleviated by the treatments described herein, this need not be the case. Even a partial or intermittent relief of symptoms may be of great benefit to the recipient. In addition, treatment of the patient is usually not a single event. Rather, a nucleic acid according to the present disclosure will likely be administered on multiple occasions, that may be, depending on the results obtained, several days apart, several weeks apart, or several months apart, or even several years apart.

The invention will be further illustrated by the following figures and examples. However, these examples and figures should not be interpreted in any way as limiting the scope of the present invention.

### EXAMPLES

### Example 1: Synthesis of Antisense Nucleic Acids Targeting Exon 73 of the Human COL7A1 Gene

The synthesis of the antisense nucleic acids targeting exon 73 of the human COL7A1 gene was performed as described in the PCT application published under n° WO 2018/193428.

### Example 2: Exon 73 Skipping Efficiency of Nucleic Acids According to the Present Disclosure

A. Materials and Methods

A.1. Dimerization Assay

10 μg of tcDNA diluted in Glycerol 50% were loaded onto a non-denaturing acrylamide-Bis 15% gel with Tris-Acetate Buffer for 2 h at 90V. Acrylamide gel was then incubated with Stains-All (Sigma-Aldrich) for 15 min with gentle shaking and image was acquired with an Epson Scan.

A.2. Skipping Efficiency 50 000 cells/wells were seeded in a 6-well plate 2 days before transfection. Three doses of AS Os were transfected (50, 100 and 200 nM) in duplicate using Lipofectamin LTX+(ThermoFischer) following manufacturer protocol. Total RNAs were extracted 72h after the transfection using the Qiagen RNeasy Mini Kit (Qiagen) following manufacturer protocol. Then 1 μg of total RNA was reverse transcribed with the SuperScript IV kit (Invitrogen) following manufacturer protocol. The first strand is then subjected to PCR using primers specific to exon boundaries 70-71 and to exon 78 of COL7A1 (Table X). The PCR products were analyzed electrophoresis on 2% agarose gel and exon skipping efficacy was assessed by densitometry quantification using the ImageLab software (Biorad).

TABLE 1
primer sequences

| Primer | Sequence | SEQ ID NO. |
|---|---|---|
| Δ73/Δ74-PCR1 FW | GAAGTGTGCCGAATGTGGAT | 7 |
| Δ73/Δ74-PCR1 Rev | CTCCCCGGTCTCCTTTGAT | 8 |

B. Results

The results are detailed in Table 2, at the end of the present disclosure.

The results of Table 2 show that all the tested nucleic acids have a good exon 73 skipping efficiency in vitro, with 50% or more efficacy.

Nucleic acids termed SY-0871_MK986 and SY-0874_MK989 induce some dimerization and are thus not optimal for an in vivo use.

Noticeably, the nucleic acid termed SY-870 does not induce any dimerization.

TABLE 2
Skipping efficiency

| | | | | Efficacy | SEQ ID |
|---|---|---|---|---|---|
| Name | Sequence | Size | Dimerization | (%) | NO. |
| SY-0869_MK984 | Palm-C6-amino -* GCCCGCGTTCTCCAG | 15 | + | 80 | 1 |
| SY-0870_MK985 | Palm-C6-amino -* GCCcGCGTTCTCCAG | 15 | | 90 | 2 |
| SY-0871_MK986 | Palm-C6-amino -* GCCCgCGTTCTCCAG | 15 | ++ | 50 | 3 |
| SY-0872_MK987 | Palm-C6-amino -* GCCCGcGTTCTCCAG | 15 | + | 60 | 4 |
| SY-0873_MK988 | Palm-C6-amino -* GCCCGCgTTCTCCAG | 15 | + | 40 | 5 |
| SY-0874_MK989 | Palm-C6-amino -* GCCCGCGTTcTCCAG | 15 | ++ | 50 | 6 |

1. A nucleic acid consisting of N consecutive nucleotides of the nucleic acid sequence of SEQ ID NO. 1 below:
(SEQ ID NO. 1)
5′-GCCCGCGTTCTCCAG-3′,
wherein,
N is an integer ranging from 13 to 15
N-x nucleotides consist of tricyclonucleotides, with x being an integer equal to 1 or 2,
x nucleotides consist of 2′-O-methyl-ribonucleotides, and
each nucleotide is linked to an adjacent nucleotide through a phosphodiester internucleoside linkage

2. The nucleic acid according to claim 1, wherein a tricylonucleotide consists of a compound of the formula (I) below: 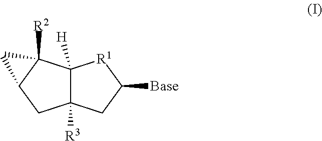
wherein
R1 means 0, S, CH 2 or NR where R is hydrogen or a (C1-C3) non-substituted alkyl,
each of R2 and R3, one independently from the other, means an internucleoside linkage, and
“Base” means a nucleobase.

2. The nucleic acid according to claim 1, wherein R1 means 0 and R2 and R3 are, one independently from the other, internucleoside linkages as defined in claim 2.

3. The nucleic acid according to any one of claims 1 and 2, which comprises one 2′-O-methyl-ribonucleotide.

4. The nucleic acid according to any one of claims 1 to 3, wherein:
N is 15,
x means 1,
all internucleoside linkages consist of phosphodiester linkages, and
the said nucleic acid comprises one nucleotide consisting of 2′-O-methyl-ribonucleotide.

5. The nucleic acid according to any one of claims 1 to 4, which consists of the oligonucleotide of SEQ ID NO. 1, wherein:
all internucleoside linkages consist of phosphodiester linkages, and
all the nucleotides consist of tricyclonucleotides, excepted the cytosyl nucleotide located at position 4, in the sense from the 5′-end to the 3′-end, which consists of a 2′-O-Methyl ribocytosyl.

6. The nucleic acid according to any one of claims 1 to 5, comprising one or more lipid moieties covalently linked thereto.

7. The nucleic acid according to claim 6, wherein each of the said lipid moieties is independently selected from the group consisting of a saturated fatty acid moiety and an unsaturated fatty acid moiety.

8. The nucleic acid according to claim 7, wherein the said one or more lipid moieties consist of a palmitoyl group.

9. The nucleic acid according to any one of claims 6 to 8, wherein the 5′ end nucleotide thereof is covalently linked to a palmitoyl group.

10. A vector comprising a nucleic acid according to nay one of claims 1 to 9.

11. A pharmaceutical composition comprising a nucleic acid according to any one of claims 1 to 9, or a vector according to claim 10, and a pharmaceutically acceptable vehicle.

12. The nucleic acid according to any one of claims 1 to 9, or the vector according to claim 10, for use as a medicament.

13. The nucleic acid according to any one of claims 1 to 9, or the vector according to claim 10, for its use for treating a subject affected with Dystrophic Epidermolysis Bullosa.

14. The nucleic acid according to any one of claims 1 to 9, or the vector according to claim 10, for use in a method for restoring the function of a mutated type VII collagen comprising the step of preventing splicing of exon 73 of the COL7A1 gene which encodes an amino acid sequence of type VII collagen involved in dysfunction of a mutated type VII collagen.

15. A method for the treatment of a patient suffering from Dystrophic Epidermolysis Bullosa comprising the step of administering to the said patient a nucleic acid according to any one of claims 1 to 9.


SEQUENCE LISTING (8 sequences)

SEQ ID NO: 1 - DNA, 15 bp, Artificial Sequence
  Synthetic antisense
  misc_feature 1: note=“tricyclonucleotide”
  misc_feature 1: note=“covalently linked to Palm-C6-Amino”
  misc_feature 2: note=“tricyclonucleotide”
  misc_feature 3: note=“tricyclonucleotide”
  misc_feature 4: note=“tricyclonucleotide”
  misc_feature 5: note=“tricyclonucleotide”
  misc_feature 6: note=“tricyclonucleotide”
  misc_feature 7: note=“tricyclonucleotide”
  misc_feature 8: note=“tricyclonucleotide”
  misc_feature 9: note=“tricyclonucleotide”
  misc_feature 10: note=“tricyclonucleotide”
  misc_feature 11: note=“tricyclonucleotide”
  misc_feature 12: note=“tricyclonucleotide”
  misc_feature 13: note=“tricyclonucleotide”
  misc_feature 14: note=“tricyclonucleotide”
  misc_feature 15: note=“tricyclonucleotide”
gcccgcgttctccag

SEQ ID NO: 2 - DNA, 15 bp, Artificial Sequence
  Synthetic antisense
  misc_feature 1: note=“tricyclonucleotide”
  misc_feature 1: note=“covalently linked to Palm-C6-Amino”
  misc_feature 2: note=“tricyclonucleotide”
  misc_feature 3: note=“tricyclonucleotide”
  misc_feature 4: note=“tricyclonucleotide”
  misc_feature 4: note=“2′-O-methyl nucleoside”
  misc_feature 5: note=“tricyclonucleotide”
  misc_feature 6: note=“tricyclonucleotide”
  misc_feature 7: note=“tricyclonucleotide”
  misc_feature 8: note=“tricyclonucleotide”
  misc_feature 9: note=“tricyclonucleotide”
  misc_feature 10: note=“tricyclonucleotide”
  misc_feature 11: note=“tricyclonucleotide”
  misc_feature 12: note=“tricyclonucleotide”
  misc_feature 13: note=“tricyclonucleotide”
  misc_feature 14: note=“tricyclonucleotide”
  misc_feature 15: note=“tricyclonucleotide”
gcccgcgttctccag

SEQ ID NO: 3 - DNA, 15 bp, Artificial Sequence
  Synthetic antisense
  misc_feature 1: note=“tricyclonucleotide”
  misc_feature 1: note=“covalently linked to Palm-C6-Amino”
  misc_feature 2: note=“tricyclonucleotide”
  misc_feature 3: note=“tricyclonucleotide”
  misc_feature 4: note=“tricyclonucleotide”
  misc_feature 5: note=“tricyclonucleotide”
  misc_feature 5: note=“2′-O-methyl nucleoside”
  misc_feature 6: note=“tricyclonucleotide”
  misc_feature 7: note=“tricyclonucleotide”
  misc_feature 8: note=“tricyclonucleotide”
  misc_feature 9: note=“tricyclonucleotide”
  misc_feature 10: note=“tricyclonucleotide”
  misc_feature 11: note=“tricyclonucleotide”
  misc_feature 12: note=“tricyclonucleotide”
  misc_feature 13: note=“tricyclonucleotide”
  misc_feature 14: note=“tricyclonucleotide”
  misc_feature 15: note=“tricyclonucleotide”
gcccgcgttctccag

SEQ ID NO: 4 - DNA, 15 bp, Artificial Sequence
  Synthetic antisense
  misc_feature 1: note=“tricyclonucleotide”
  misc_feature 1: note=“covalently linked to Palm-C6-Amino”
  misc_feature 2: note=“tricyclonucleotide”
  misc_feature 3: note=“tricyclonucleotide”
  misc_feature 4: note=“tricyclonucleotide”
  misc_feature 5: note=“tricyclonucleotide”
  misc_feature 6: note=“tricyclonucleotide”
  misc_feature 6: note=“2′-O-methyl nucleoside”
  misc_feature 7: note=“tricyclonucleotide”
  misc_feature 8: note=“tricyclonucleotide”
  misc_feature 9: note=“tricyclonucleotide”
  misc_feature 10: note=“tricyclonucleotide”
  misc_feature 11: note=“tricyclonucleotide”
  misc_feature 12: note=“tricyclonucleotide”
  misc_feature 13: note=“tricyclonucleotide”
  misc_feature 14: note=“tricyclonucleotide”
  misc_feature 15: note=“tricyclonucleotide”
gcccgcgttctccag

SEQ ID NO: 5 - DNA, 15 bp, Artificial Sequence
  Synthetic antisense
  misc_feature 1: /note=“tricyclonucleotide”
  misc_feature 1: note=“covalently linked to Palm-C6-Amino”
  misc_feature 2: note=“tricyclonucleotide”
  misc_feature 3: note=“tricyclonucleotide”
  misc_feature 4: note=“tricyclonucleotide”
  misc_feature 5: note=“tricyclonucleotide”
  misc_feature 6: note=“tricyclonucleotide”
  misc_feature 7: note=“tricyclonucleotide”
  misc_feature 7: note=“2′-O-methyl nucleoside”
  misc_feature 8: note=“tricyclonucleotide”
  misc_feature 9: note=“tricyclonucleotide”
  misc_feature 10: note=“tricyclonucleotide”
  misc_feature 11: note=“tricyclonucleotide”
  misc_feature 12: note=“tricyclonucleotide”
  misc_feature 13: /note=“tricyclonucleotide”
  misc_feature 14: /note=“tricyclonucleotide”
  misc_feature 15: /note=“tricyclonucleotide”
gcccgcgttctccag

SEQ ID NO: 6 - DNA, 15 bp, Artificial Sequence
  Synthetic antisense
  misc_feature 1: note=“tricyclonucleotide”
  misc_feature 1: note=“covalently linked to Palm-C6-Amino”
  misc_feature 2: note=“tricyclonucleotide”
  misc_feature 3: note=“tricyclonucleotide”
  misc_feature 4: note=“tricyclonucleotide”
  misc_feature 5: note=“tricyclonucleotide”
  misc_feature 6: note=“tricyclonucleotide”
  misc_feature 7: note=“tricyclonucleotide”
  misc_feature 8: note=“tricyclonucleotide”
  misc_feature 9: note=“tricyclonucleotide”
  misc_feature 10: note=“tricyclonucleotide”
  misc_feature 10: note=“2′-O-methyl nucleoside”
  misc_feature 11: note=“tricyclonucleotide”
  misc_feature 12: note=“tricyclonucleotide”
  misc_feature 13: note=“tricyclonucleotide”
  misc_feature 14: note=“tricyclonucleotide”
  misc_feature 15: note=“tricyclonucleotide”
gcccgcgttctccag

SEQ ID NO: 7 - DNA, 20 bp, Artificial Sequence
  Synthetic Primer
gaagtgtgccgaatgtggat

SEQ ID NO: 8 - DNA, 19 bp, Artificial Sequence
  Synthetic Primer
ctccccggtctcctttgat